(19) United States
(12) Patent Application Publication
(10) Pub. No.: US 2009/0220935 A1
(43) Pub. Date: Sep. 3, 2009

(54) APPARATUS AND METHOD FOR DISSOLVED OXYGEN CONTROL IN PARALLEL INTEGRATED BIOREACTOR ARRAY

(75) Inventors: Harry Lee, Boston, MA (US); Kevin Shao-Kwan Lee, Cambridge, MA (US)

Correspondence Address: CHOATE, HALL & STEWART LLP, TWO INTERNATIONAL PLACE, BOSTON, MA, 02110, US

(73) Assignee: Massachusetts Instutite of Technology, Cambridge, MA (US)

(21) Appl. No.: 12/281,919

(22) PCT Filed: Mar. 9, 2007
(86) PCT No.: PCT/US07/06246

§ 371 Date: Mar. 9, 2009

Related U.S. Application Data
(60) Provisional application No. 60/780,982, filed on Mar. 10, 2006.

(51) Int. Cl. C12Q 3/00; C12M 1/00; C40B 40/00

(52) U.S. Cl. 435/3; 435/297.1; 506/13

(57) ABSTRACT

Parallel, integrated bioreactor. The bioreactor includes a plurality of growth chambers, each growth chamber associated with a peristaltic oxygenating mixer and separated therefrom by a porous membrane. Each oxygenating mixture has a gas inlet and a gas outlet with the gas inlet in fluid communication with a gas reservoir. A gas mixer switch is provided for controlling oxygen concentration in the reservoir. The apparatus and methods disclosed in the application allow precise control over dissolved oxygen concentration with a quick response time.

This application claims priority to provisional application Ser. No. 60/780,982 filed Mar. 10, 2006 the contents of which are incorporated herein by reference.

### BACKGROUND OF THE INVENTION

This invention relates to bioreactors and more particularly to microbioreactor arrays with dissolved oxygen control.

Commonly used microbial cell culture tools have remained largely unchanged for the last few decades. Microbiologists have long had to compromise between the throughput and capabilities of the shaken microtiter plate, shake flasks, and the stirred tank bioreactor. While conventional high throughput methods, such as shaken microtiter plates or flasks, offer the advantage of high parallelism, they suffer from a lack of control over pH and low oxygen transfer capacity. In contrast, bench scale stirred tank bioreactors offer full control over culture conditions and high oxygen transfer capacity; however, these systems are labor intensive, complicated and costly to operate in parallel. For applications such as industrial bioprocess development or scientific studies of microbial cells at high cell density, both high-throughput and the capacity for high cell density growth under controlled conditions are desirable.

Recent efforts to address the need for a parallel bioreactor system with the capabilities of a stirred tank reactor have focused on improving the oxygen transfer rate of microtiter plates, improving the control capabilities of shake flasks, improving the parallelism of stirred tank bioreactors, or developing microfabricated bioreactor systems. Each of these approaches has addressed parallelism, oxygenation, control, automation, and scalability to various degrees. Of these approaches, miniature arrays of stirred tanks with robotic fluid handling have achieved the highest level of performance in terms of cell density and control parameters. However, these systems require expensive pipetting robotics and careful sterilization of the pipette tips to prevent contamination during frequent sampling.

A lab-on-a-chip approach offers the potential for circumventing the need for robotic multiplexing. However, none of the microbioreactor systems developed to date have taken advantage of microfluidic integration to achieve parallelism. In addition, no existing lab-on-a-chip approach has succeeded, even in a single reactor, in providing the oxygen transfer rate and pH control capabilities of stirred tank bioreactors that are required for high cell density growth. See, “Microbioreactor arrays with integrated mixers and fluid injectors for high throughput experimentation with pH and dissolved oxygen control” by Harry L. T. Lee et al. The Royal Society of Chemistry 2006, Lab Chip, 2006, 6, 1229-1235. The contents of this paper and its electronic supplementary information are incorporated in their entirety herein by reference.

It is therefore an object of the invention to provide a parallel integrated bioreactor array that enables control of dissolved oxygen concentration rather than merely providing a high oxygen transfer rate.

### SUMMARY OF THE INVENTION

The parallel, integrated bioreactor of the invention includes a plurality of growth chambers, each growth chamber associated with a peristaltic oxygenating mixer including an oxygen permeable membrane. Each of the oxygenating mixers has a gas inlet and a gas outlet, the gas inlet being in fluid communication with a gas reservoir. A gas mixer switch is provided for controlling oxygen concentration in the reservoir. In a preferred embodiment, the bioreactor further includes pressurisible valves for controlling the gas inlets and outlets. In an embodiment, the bioreactor includes a first layer having a plurality of growth chambers, a second layer including the peristaltic oxygenating mixer and a third layer including a plurality of pressurisible valves. In a preferred embodiment there is provided a source of air and a source of oxygen. The gas mixer switch alternatingly connects the air source and the oxygen source to the reservoir during a selected duty cycle to control oxygen concentration in the reservoir.

In another aspect, the invention is a method for controlling dissolved oxygen concentration in a vessel comprising controlling oxygen concentration in a gas reservoir by switching an inlet to the reservoir between a gas having a relatively lower concentration of oxygen and a gas having a relatively higher concentration of oxygen. The gas is delivered from the gas reservoir to the vessel and dissolved oxygen concentration in the vessel is sensed. The switching of the inlet in response to the sensed dissolved oxygen concentration is controlled so as to control dissolved oxygen concentration in the vessel. One example of a control algorithm is a PID control algorithm in which the duty cycle of the gas mixing switch is set according to the dissolved oxygen concentration error, the integral of the error, and the derivative of the error. Such a control algorithm is commonly known in the art.

In yet another aspect, the invention allows control over the pressurization rate of the mixer membrane sections. Control of the pressurization rate is accomplished by restricting the air flow into a cavity on the gas side of the membrane section and enlarging the volume of the cavity on the gas side of the membrane section. Restricting air flow can be accomplished by reducing the cross-sectional area of the gas inlet channel. A larger air flow resistance increases the membrane pressurization time. Further, a larger volume cavity on the gas side of the membrane section increases the membrane pressurization rate.

According to another aspect, a minimal number of pneumatic pressure signals for the membrane valves are required to actuate the membrane sections of all of the mixer membranes across different vessels. It is preferred that the number of pneumatic pressure signals equals the number of distinct membrane section pressurization patterns.

The microfabricated integrated design disclosed herein is a fundamentally different approach that provides a less expensive, higher performance system than achievable based on conventional methods. The apparatus and methods disclosed herein allow precise control over dissolved oxygen concentration with a quick response time. The devices disclosed herein do not need to make use of expensive mass flow controllers that have to be carefully calibrated nor does the present invention use adjustment of agitation (stirring) speed to increase oxygen transport.

### BRIEF DESCRIPTION OF THE DRAWING

FIG. 1 is a schematic illustration of the switching system for controlling oxygen concentration according to one embodiment of the invention.

FIG. 2 is a schematic illustration of an array of four growth chambers having independent oxygen concentration control.

FIG. 3 is a cross sectional view of one of the microreactors in FIG. 2.

FIG. 4 is schematic illustration of another embodiment of the invention.

FIG. 5 is a cross-sectional view of the embodiment shown in FIG. 4.

### DESCRIPTION OF THE PREFERRED EMBODIMENTS

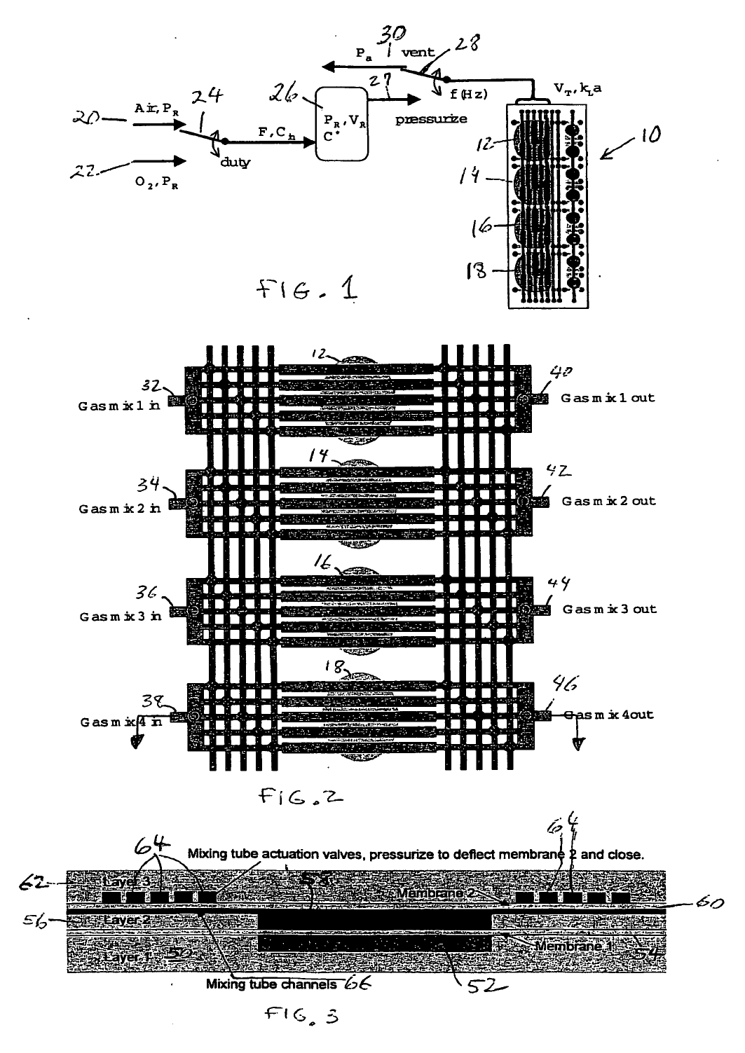

With reference first to FIG. 1 a microbioreactor array 10 includes four integrated bioreactors 12, 14, 16 and 18. Each of the bioreactors 12-18 includes a growth chamber for microbial cell culture.

The microbioreactor array 10 is connected to a system illustrated in the left hand portion of FIG. 1 for controlling dissolved oxygen concentration. A source of air 20 and a source of oxygen 22 are in fluid communication with a movable conduit or switch 24 that alternately connects either the source of air 20 or the source of oxygen 22 to a reservoir 26. The conduit 24 may be cycled at an exemplary frequency between 0.1 and 3 Hz. The duty cycle of the conduit switching 24 determines the oxygen concentration in the reservoir 26. That is, the oxygen concentration is a function of the amount of time that the conduit 24 is in fluid communication with the air source 20 as compared with the amount of time it is in fluid communication with the source of oxygen 22. The oxygen concentration may be expressed by the following equation if the gas pressure at the normally closed port is the same as the gas pressure at the normally open port:

Cₒᵤₜ=C_{normally closed}*d+C_{normally open}*(1−d).

In this equation, Cₒᵤₜ is the oxygen concentration at the outlet 27 of the reservoir 26, d is the duty cycle, C_{normally closed} is the concentration of gas at the normally closed port 22, and C_{normally open} is the concentration of oxygen at the normally open port 20. For example, if the duty cycle is 25%, the concentration at the output of the reservoir 26 is 500%*0.25+100%*0.75=200% if the normally closed port 22 is connected to pure oxygen and the normally open port 20 is connected to air.

A second switching unit 28 is either in fluid communication with the output of the reservoir 26 or in fluid communication with a vent to the atmosphere 30. When the unit 28 is in fluid communication with the output of the reservoir 26, the output of the reservoir 26 pressurizes the mixing and oxygenation structures of the microbioreactor array 10. The mixing and oxygenation structures are the vertical structures that cross the four bioreactor growth chambers 12-18 in FIG. 1 or the horizontal structures that cross each growth well in FIG. 2. It is preferred that the movable conduit 24 be controlled by a 3-way solenoid switch (not shown) (an example is the Lee Company part number LHDA0521111H). In this embodiment, air from the source 20 is connected to a normally open port of the solenoid switch and pure oxygen from the source 22 is connected to a normally closed port and the reservoir 26 is connected to the common port.

This method of controlling oxygen concentration is most appropriate for closed loop control of oxygen concentration because the actual concentration may be very sensitive to the detailed performance of the switch and the gas pressures at the normally open and normally closed ports. With closed loop control, based on the error between the desired oxygen concentration, and the oxygen concentration measured within the growth chamber, the duty cycle is set such that the output oxygen concentration is at a desired level, independent of the detailed operation of the switch or gas pressures. The configuration shown in FIG. 1 is capable of maintaining a minimum dissolved oxygen of the 4 integrated bioreactors 12-18 above a threshold dissolved oxygen concentration. Independent control is not possible in this embodiment because the mixing and oxygenation structures for each bioreactor are not isolated.

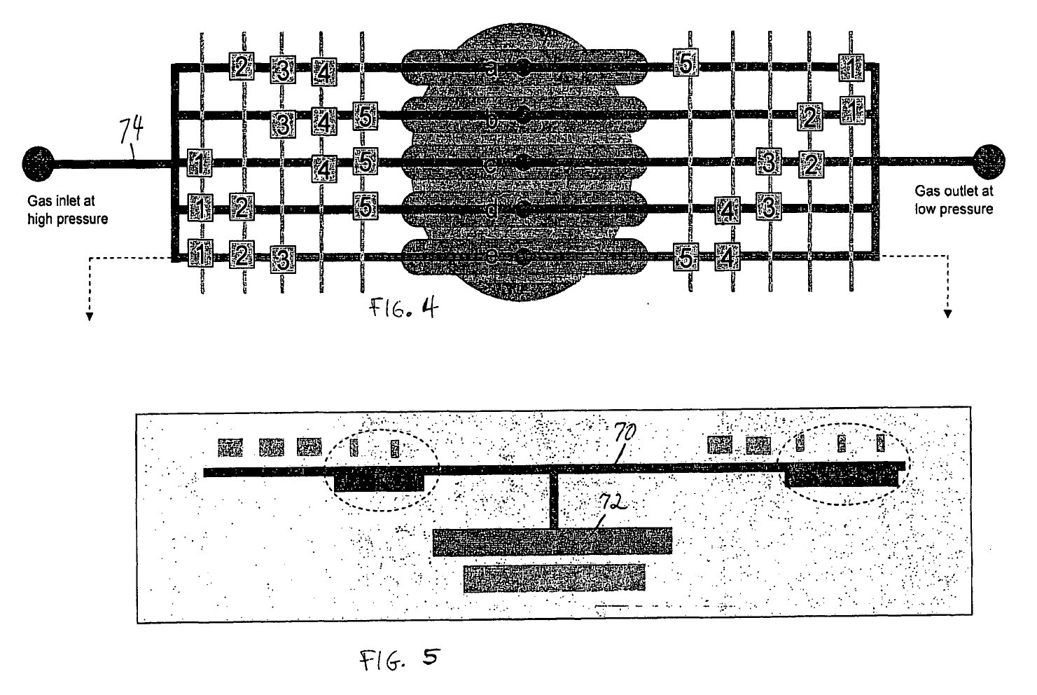

Embodiments of the invention illustrated in FIGS. 2 and 3 permit independent control over dissolved oxygen in each of the bioreactors. As shown in FIG. 2 each of the bioreactors 12, 14, 16 and 18 have gas inlets 32, 34, 36 and 38 and gas outlets 40, 42, 44 and 46 that are isolated from each other. As will be apparent to those of skill in the art, each of the inlets 32, 34, 36 and 38 will be connected to the outlet 27 of an independent set of reservoirs 26, each with its own gas mixing switch. In this way, the oxygen concentration entering the bioreactors 12-18 can be independently controlled.

As shown in FIG. 3, each bioreactor in the array shown in FIG. 2 is made of a multilayer structure. A first layer 50 includes a growth well or chamber 52. A membrane 54 separates the layer 50 from a second layer 56 that includes a cavity 58 defining a peristaltic mixing tube. A second membrane 60 separates the layer 56 from a third layer 62 that includes pressurisible actuation valves 64.

Closed actuation valves 64 are marked by an X in FIG. 2. The X's in FIG. 2 are not representative of the open/closed state of a device with four bioreactors in operation. In operation, all of the valves associated with a common (vertical) actuation line would be closed or open together. In other words, all of the circles along a given actuation line would have an X or not. The X's in FIG. 2 should be interpreted as showing different pressurization states of the mixing tubes for a single bioreactor at different times. As will become clear below, the pressurization of the mixing tube actuation valves 64 are shared such that applying pressure to a single valve actuation line can actuate multiple valves across the different bioreactors, and therefore control the pressurization state of the mixing tubes 58, for each bioreactor. As can be seen in FIG. 3 mixing tube channels 66 may be sealed shut when the actuation valves 64 are pressurized. For a given mixing tube, opening a valve at the gas inlet side of the mixing tube and closing a valve at the gas outlet side pressurizes the tube, and closing the valve at the gas inlet side and opening the valve at the gas outlet side vents the tube. By opening and closing these valves in sequence, the mixing tube pressurization pattern can be made to approximate peristalsis. In this way, pressurization of membrane sections in a pre-determined sequence causes the contents of the vessel to be mixed and improves the efficiency of oxygen transfer.

With reference again to FIG. 2, each bioreactor 12-18 has an independent gas mixture inlet and gas mixture outlet. The gas mixture inlet is at a higher pressure than ambient, e.g., approximately 4 psi. The gas mixture outlet is at approximately ambient pressure. The deflection of the peristaltic mixing tubes 58 can be controlled by opening and closing the valves 64 (x in FIG. 2). For illustrative purposes only, for bioreactor 12, the top two tubes would be pressurized, for bioreactor 14, the second and third tubes would be pressurized, for bioreactor 16, the third and fourth tubes would be pressurized, and for bioreactor 18, the fourth and fifth tubes would be pressurized. In actual operation, the pressurization state of the tubes for each device would be the same because the actuation of the mixing tube actuation valves would be shared.

With reference again to FIG. 1, it is to be understood that the oxygen source 22 may in fact include oxygen enriched air rather than strictly pure oxygen. Similarly, the source of air 20 may be oxygen neutral or oxygen deficient air.

Another embodiment of the invention is shown in FIG. 4. In this embodiment, the square boxes including numbers represent normally open valves that can close an underlying channel 70 as shown in FIG. 5. The channel 70 is in fluid communication with a region 72. Valves with the same number are activated by the same control signal. For example, activating control signal 1 pressurizes membrane sections a and b only. Activating control signal 2 pressurizes membrane sections b and c only. Activating control signal 3 pressurizes membrane sections c and d only. Similarly, activating control signal 4 pressurizes membrane sections d and e only. Finally, activating control signal 5 pressurizes membrane sections a and e only. Therefore, only 5 valve control signals are required to generate the 5 membrane section pressurization states. If the valves are pneumatic membrane pinch valves, an underlying channel can always be kept open regardless of the pneumatic control signal by enlarging the underlying channel as indicated by the dashed ovals. Such enlargement prevents the membrane from sealing the channel closed.

The architecture illustrated in the embodiments of FIGS. 4 and 5 illustrate that the number of pressure control signals can equal the number of distinct membrane section pressurization patterns. For each pressurization pattern (for example, the first two membrane sections a and b are pressurized and the remaining membrane sections are unpressurized), there is a predetermined configuration of closed valves that will place the pressurized membrane sections in fluid communication with the inlet and isolate them from the outlet, and place the remaining membrane sections in fluid communication with the outlet and isolate them from the inlet. The valves are arranged to be controlled by a common actuation signal (the gas side of the membrane pinch valves are in fluid communication). Therefore, one actuation signal can generate a pressurization pattern. Because the valves are normally open, multiple pre-determined patterns can be arranged to control the fluid communication between the membrane sections and the fluid inlet and outlet wherein each valve pattern, when actuated, generates a membrane pressurization pattern. Unactuated valve patterns do not effect the pressurization state of the membrane sections.

It should be understood that the single bioreactor configuration shown in FIG. 4 can be repeated multiple times to form an array similar to what is shown in FIG. 2. In such a configuration, the single actuation signal that would generate a pressurization pattern in one bioreactor would be shared by one or more of the bioreactors in the array. Therefore, the number of actuation signals could remain the same for any number of bioreactors in the array. This property allows advantageous scaling of the number of bioreactors in the array without requiring an increase in the number of actuating signals.

FIGS. 4 and 5 also illustrate an aspect of the invention that allows control over the pressurization rate of the mixer membrane sections. Such control is important to control the fluid mechanical forces generated by a deflecting membrane. Control of the pressurization rate can be accomplished by restricting the air flow into the cavity on the gas side of the membrane section and enlarging the volume of the cavity on the gas side of the membrane section. Restricting the air flow can be accomplished by reducing the cross-sectional area of the gas inlet channel 74. A larger air flow resistance increases the membrane pressurization time. Also, a larger volume cavity 72 on the gas side of the membrane section increases the membrane pressurization rate.

It is recognized that modifications and variations of the invention disclosed herein will be apparent to those of ordinary skill in the art and it is intended that all such modifications and variations be included within the scope of the appended claims.

What is claimed is:

1. Parallel, integrated bioreactor comprising:
a plurality of growth chambers, each growth chamber associated with a peristaltic oxygenating mixer including an oxygen permeable membrane;
each said oxygenating mixer having a gas inlet and a gas outlet, the gas inlet in fluid communication with at least one gas reservoir; and
a gas mixer switch for controlling oxygen concentration in each gas reservoir.

2. The bioreactor of claim 1 further including pressurisible valves for controlling the gas inlets and outlets.

3. The bioreactor of claim 2 wherein the bioreactor comprises a first layer including a plurality of said growth chambers, a second layer including the peristaltic oxygenating mixer, and a third layer including a plurality of pressurisible valves.

4. The bioreactor of claim 1 further including a source of air and a source of oxygen, wherein the gas mixer switch alternatingly connects the air source and oxygen source to the reservoir during a selected duty cycle to control oxygen concentration in the reservoir.

5. The bioreactor of claim 1 wherein the oxygenating mixer is operated in a way to mimic peristalsis.

6. The bioreactor of claim 1 wherein a minimal number of pressure signals for membrane valves are required to actuate membrane sections of all of the mixer membranes across different vessels.

7. The bioreactor of claim 6 wherein the number of pressure signals equals the number of distinct membrane section pressurization patterns.

8. The bioreactor of claim 1 further including means to control pressurization rate of mixer membrane sections.

9. The bioreactor of claim 8 wherein control over pressurization rate is effected by restricting air flow into a cavity on the gas side of a membrane section and enlarging the volume of the cavity on the gas side of the membrane section.

10. Method for controlling dissolved oxygen concentration in a vessel comprising:
controlling oxygen concentration in a gas reservoir by switching an inlet to the reservoir between a gas having a relatively lower concentration of oxygen and a gas having a relatively higher concentration of oxygen;
delivering gas from the gas reservoir to the vessel;
sensing dissolved oxygen concentration in the vessel; and
controlling the switching of the inlet in response to the sensed dissolved concentration thereby to control dissolved oxygen concentration in the vessel.

11. The method of claim 10 wherein the delivering step causes contents of the vessel to be mixed to improve efficiency of oxygen transfer.

12. An array of a plurality of bioreactors wherein a single actuation signal generates a pressurization pattern in each bioreactor in the array such that the number of actuation signals remains the same independent of the number of bioreactors in the array.
